(19) United States
(12) Patent Application Publication
(10) Pub. No.: US 2013/0041354 A1
(43) Pub. Date: Feb. 14, 2013

(54) REGISTERING OCT OR OTHER EYE MEASUREMENT SYSTEM WITH A FEMTOSECOND FLAP CUT OR OTHER LASER SURGICAL TREATMENT USING A COMMON PATIENT INTERFACE

(71) Applicants: AMO Development, LLC., Santa Ana, CA (US); Andrew Y. Pang, (US); Andrew Y. Pang, (US)

(72) Inventors: Michael Brownell, San Clemente, CA (US); Tibor Juhasz, Irvine, CA (US)

(73) Assignee: AMO Development, LLC., Santa Ana, CA (US)

(21) Appl. No.: 13/629,269

(22) Filed: Sep. 27, 2012

Related U.S. Application Data
(63) Continuation of application No. 12/714,146, filed on Feb. 26, 2010.
(60) Provisional application No. 61/155,903, filed on Feb. 26, 2009.

(51) Int. Cl. A61F 9/009

(52) U.S. Cl. 606/4

(57) ABSTRACT

Methods and systems for ablating internal targets in the eye of a patient employ an ophthalmic measurement system to acquire location data of structures in the eye. A controller calculates target locations based on the location data received from the ophthalmic measurement system, and a laser emits a laser beam to treat the target locations received from the controller. A patient interface is attached to the eye to provide a common reference surface for both the laser and the ophthalmic measurement system. The patient interface may engage the eye around the optically used cornea, and without conforming the optically used cornea to a predetermined shape.

### CROSS-REFERENCE TO RELATED APPLICATION

This application is a continuation of prior U.S. application Ser. No. 12/714,146, filed Feb. 26, 2010, which claims the benefit of U.S. Provisional Application No. 61/155,903, filed Feb. 26, 2009.

### BACKGROUND OF THE INVENTION

1. Field of the Invention

The field of the present invention is generally related medical devices, systems, and methods for their use, typically for measuring and/or treating tissues of an eye and, more particularly, to provide a common reference structure from which to base measurements of internal tissues of the eye, and from which to direct treatments toward selected targets so as to correct refractive defects of the eye, treat ophthalmic disease states, and/or the like.

2. Background

Various laser procedures or operations benefit from a laser beam that is properly directed to a specific target within the patient's eye. For example, in an ophthalmic laser surgery where eye tissue is to be photoaltered, the post-treatment quality of the patient's vision may largely depend on correct targeting of the laser beam. Such ophthalmic surgical procedures can rely on laser targets on or in the cornea, sclera, iris, eye lens, capsular bag and other structures of the eye. A precision targeting of the laser beam is also beneficial in many non-ophthalmic laser procedures. Existing laser eye surgery systems do a very good job of directing the laser beam toward the intended targets.

Along with the accuracy of the targeting and beam directing systems, modern laser eye treatment systems benefit from high-quality measurement data. A variety of specialized diagnostic tools have been developed to facilitate highly accurate refractive prescriptions to be developed. In particular, wavefront aberrometers have recently revolutionized laser eye surgery by providing accurate and practical measurements of the high-order refractive defects throughout the optical system. This has allowed customized photoalteration shapes or prescriptions to be derived that address the specific defects of a particular patient's eye. The combination of wavefront aberrometry and customized laser treatments can often provide final visual acuities of better than 20/20 for many patients. Such highly advantageous outcomes may be more common when the relationship between the eye measurement data and the position of the eye during treatment is known quite accurately.

For many patients, the refractive laser treatment is directed to an interior stromal tissue within a patient's cornea. In LASIK, that stromal tissue is accessed by cutting and displacing a thin flap from the anterior corneal surface, with the cut optionally being performed using a femtosecond laser. One way to accurately position the eye relative to the femtosecond laser system is to use a contact lens to shape and stabilize the eye. The position of the contact lens (typically a flat or curved glass plate referred to as an “applanation lens”) relative to the laser system is generally sufficiently known for the accurate targeting of the laser light beam, as it can be mounted to the laser system. When a flat applanation lens is used, it engages the cornea so that the applanation lens flattens the eye, thus creating a reference surface where the lens and the eye contact. Curved applanation lenses create a similarly conformed curved reference surface. The desired flap can then be cut largely by targeting a flap surface at a fixed depth into the cornea from the applation lens surface.

Femtosecond cutting of LASIK flaps and customized laser eye surgery provide great benefits for many patients. However, as with all successes, still further improvements might be desirable. Some patients with thinner corneas are not good candidates for LASIK, depending on the depth of the flap and/or the depth of tissue to be photoaltered in the refractive correction. Unfortunately, flattening the eye also can increase intraocular pressure. In some cases, applanation can result in patient gray-out and black-out in the applanated eye, and can cause ripples in deep lamellar cuts. Curved applanation lenses that more closely approximate the shape of the eye can be used, but may also not be a good fit with all corneas, may complicate scanning patterns, and/or may add cost and complexity to the surgery system. Both curved and flat applanation lenses may also displace internal structures within the eye. Hence, extension of the known ophthalmic measurement and treatment techniques and tools to allow improved ophthalmic therapies may benefit from improved systems and methods.

Thus, a need remains for the systems and methods which can target the laser light to the patient eye for the photoalteration of the eye tissue without the shortcomings of the present devices.

### SUMMARY OF THE INVENTION

Embodiments of the present invention provide systems and methods for laser therapies directed to internal targets in the eye of a patient. Typically, an ophthalmic measurement system acquires location data of one or more structures in the eye. A controller often calculates the target locations based on the location data received from the ophthalmic measurement system, and a laser emits a laser beam to ablate, photoalter, or otherwise treat the target locations received from the controller (for instance, portions of the cornea, lens, capsular bag, or another structure of the eye). A common reference surface is provided for the laser system and the ophthalmic measurement system, with the common reference surface typically being included in a patient interface that is attached to the eye using a suction ring or the like. The laser and the ophthalmic measurement system can couple with the patient interface sequentially, or the measurement and laser systems can be integrated into an overall diagnostic and photoalteration assembly which couples with the patient interface. The patient interface may engage the eye outside the optically used cornea, and without conforming the optically used cornea to a predetermined shape.

Various laser sources may be used with the inventive method and system, including infrared, visible, and UV lasers. Further, the laser sources used with the inventive methods and systems may be a continuous wave, Q-switched pulse, or mode-locked ultrashort pulse lasers, including femtosecond or picosecond ranges of light pulse duration. Some examples of the ophthalmologic measurement system are an optical coherence tomographer (OCT), a wavefront aberrometer, and a topographer.

In one embodiment, a laser surgery system for treatment of the eye has a patient interface with a reference surface and an eye-engagement surface configured to attach with the eye; an ophthalmic measurement system that, in use, generates location data corresponding to internal surfaces of the eye, where the measurement system is coupleable with the reference surface; a laser that is coupleable with the reference surface; and a controller that is coupleable with the ophthalmic measurement system and the laser. The controller is configured to process location data from the ophthalmic measurement system and to compute laser target data so that the laser photoalters an internal target within the eye in response to the location data.

In one aspect, the eye engagement surface is a substantially annular area outside of a treated area of the eye, thus inhibiting distortion of the internal target within the eye.

In another aspect, the patient interface system further has a substantially planar or spherical lens for contacting the eye. The lens conforms a central cornea of the eye to a substantially planar or spherical shape.

In yet another aspect, the ophthalmologic measurement system is an optical coherence tomographer, a wavefront aberrometer, a topographer, or a combination thereof.

In another aspect, the coupling of the ophthalmic measurement system and the laser with the reference surface is performed sequentially.

In yet another aspect, the ophthalmic measurement system and the laser are housed in a photoalteration apparatus capable of coupling with the reference surface, thus referencing the ophthalmic measurement system and the laser with the reference surface simultaneously.

In another embodiment, a system for laser surgery treatment of the eye which, in use, generates a location data corresponding to internal surfaces of the eye using an ophthalmic measurement system, which calculates internal targets within the eye based on the location data using a controller, and which photoalters the internal targets within the eye using a laser has a patient interface with a reference surface configured to couple with the laser and the ophthalmic measurement system, and an eye-engagement surface configured to attach with the eye.

In yet another embodiment, a method for laser surgery treatment of the eye has the steps of engaging a patient interface with the eye of a patient, the patient interface having a reference surface; coupling an ophthalmic measurement system with the reference surface; generating location data corresponding to internal surfaces of the eye using the ophthalmic measurement system; coupling the measurement system with a controller so that the controller computes an internal target within the eye in response to the location data; coupling the laser with the reference surface; coupling the laser with the controller; and ablating the internal target with the laser light.

In one aspect, the location data corresponding to internal surfaces of the eye are periodically refreshed, thus enabling the internal target within the eye to be refreshed.

In another aspect, the patient interface is discarded after every use.

### BRIEF DESCRIPTION OF THE DRAWINGS

One or more exemplary embodiments of the present invention will hereinafter be described in conjunction with the following drawings, wherein like reference numerals denote like components:

FIG. 1 shows a schematic view of the laser surgery system attached to the patient interface;

FIGS. 2A-C illustrate several views of the patient interface;

FIGS. 3A-C illustrate one embodiment of the laser surgery system attachment and operation;

FIG. 4 is a flowchart illustrating a method that can be used in conjunction with the system of FIGS. 3A-C;

FIGS. 5A and 5B illustrate another embodiment of the laser surgery system attachment and operation; and

FIG. 6 is a flowchart illustrating a method that can be used in conjunction with the system of FIGS. 5A-B.

### DETAILED DESCRIPTION

Embodiments of the present invention can be used to direct laser light to the target areas in the patient's eye. The laser light photoalters the target areas in the eye, for example, portions of the cornea or eye lens, in order to improve vision of the patient, or to provide an access for a subsequent surgery by cutting a flap in the cornea or opening an aperture in the capsular bag. The target areas for the laser photoalteration are calculated by a controller based on the location data received from an ophthalmic measurement system, for example, an optical coherence tomographer, a wavefront aberrometer, or a topographer. A patient interface attached to the patient's eye provides a common reference surface for the attachment of the laser and the ophthalmic measurement system.

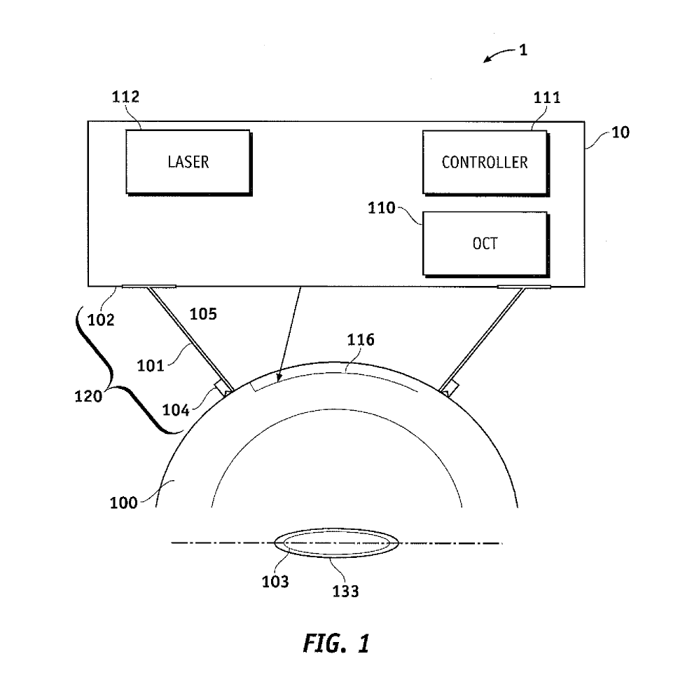

Referring now to FIG. 1, a schematic view of one embodiment of a system for laser surgical treatment according to the present invention is depicted. The system 1 has a patient interface (PI) 120 and a photoalteration apparatus 10. The patient interface 120 has a patient interface cone 101, which attaches to a patient's eye using vacuum ring 104. The outside surface of the patient interface cone 101 is in a non-slipping contact with the vacuum ring. Therefore, since the vacuum ring 104 is fixed to the patient eye, so is the patient interface cone 101. The patient interface 120 has a reference surface 102 on the side opposite from the vacuum ring 104. When attached to the patient's eye 100 and not otherwise constrained, the vacuum ring 104 and, consequently, the patient interface 120 follow the movements of the eye. Thus, the reference surface 102 of the patient interface 120 stays in an approximately fixed position to the relevant structures of the eye (cornea, eye lens, capsular bag, etc.).

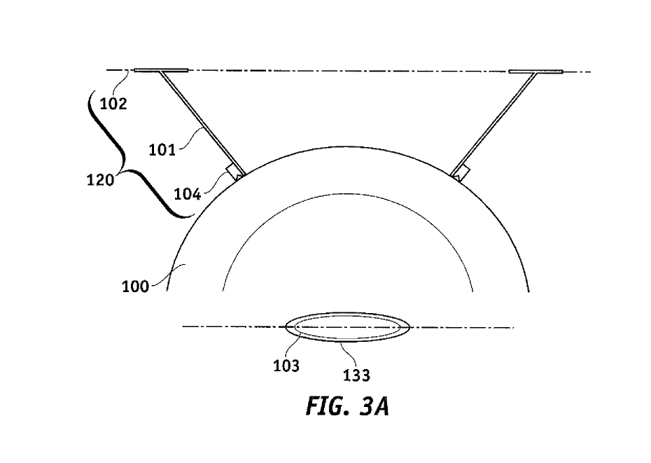

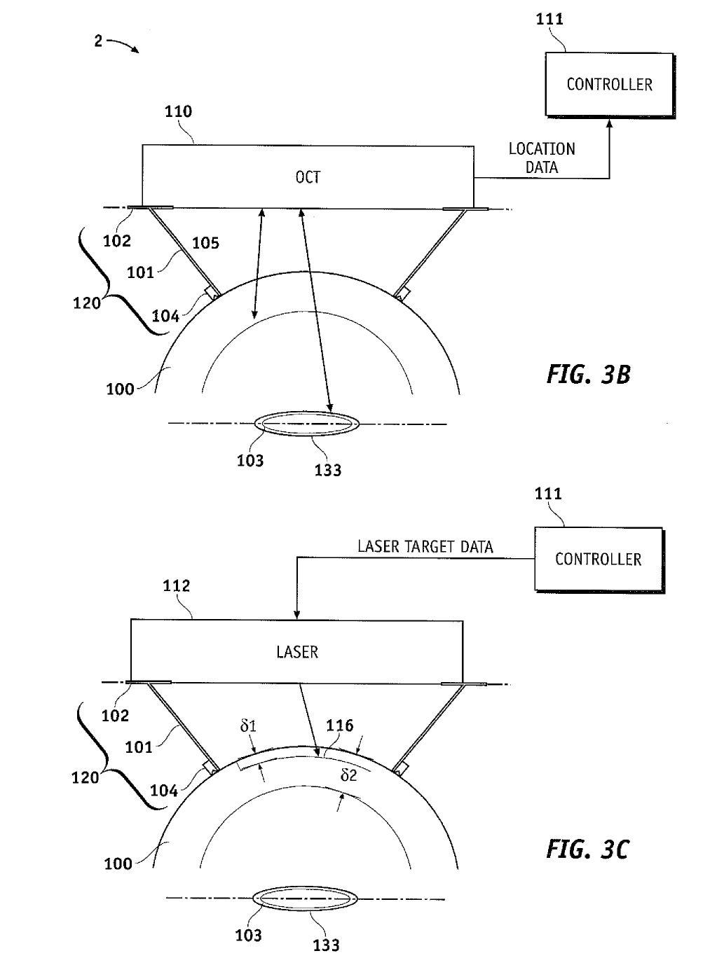

The photoalteration apparatus 10 has an ophthalmic measurement system, a controller, and a laser. Docking the photoalteration apparatus 10 to the reference surface 102 can simultaneously register the ophthalmic measurement system and the laser to the reference surface 102. A sequential docking of the ophthalmic measurement system and the laser to the reference surface is also possible, as explained below with reference to FIGS. 3A-C. The ophthalmic measurement system, which can be an optical coherence tomographer 110, acquires location data on the structures of interest in the patient's eye, and provides the data to the controller 111, which calculates the target data. The laser 112 photoalters volumes in the eye corresponding to the target data. A corneal flap 116 is an example outcome of the laser light photoalteration, but other examples are possible, for instance changing the thickness or shape of the cornea, making a flap or aperture in the capsular bag 133, or ablating portions of the eye lens 103.

The laser 112 provides a pulsed laser beam for photoalteration via a chirped pulse laser amplification system, such as described in U.S. Pat. No. RE37,585, for example. U.S. Pat. Publication No. 2004/0243111 also describes other methods of photoalteration, the entire disclosures of which are incorporated herein. Other devices or systems may be used to generate pulsed laser beams. For example, non-ultraviolet (UV), ultrashort pulsed laser technology can produce pulsed laser beams having pulse durations measured in femtoseconds. Some of the non-UV, ultrashort pulsed laser technology may be used in ophthalmic applications. For example, U.S. Pat. No. 5,993,438 discloses a device for performing ophthalmic surgical procedures to effect high-accuracy corrections of optical aberrations. U.S. Pat. No. 5,993,438, the entire disclosure of which is incorporated herein, discloses an intrastromal photodisruption technique for reshaping the cornea using a non-UV, ultrashort (e.g., femtosecond pulse duration), pulsed laser beam that propagates through corneal tissue and is focused at a point below the surface of the cornea to photodisrupt stromal tissue at the focal spot. Focusing optics preferably direct the pulsed laser beam toward the eye for plasma mediated (e.g., non-UV) photodisruption of tissue.

The pulsed laser beam has physical characteristics similar to those of the laser beams generated by a laser system disclosed in U.S. Pat. No. 4,764,930, the entire disclosure of which is incorporated herein, U.S. Pat. No. 5,993,438, or the like. For example, a non-UV, ultrashort pulsed laser beam is produced for use as an incising laser beam. This pulsed laser beam preferably has laser pulses with durations as long as a few nanoseconds or as short as a few femtoseconds. For photodisruption of the tissue, the pulsed laser beam has a wavelength that permits the pulsed laser beam to pass through the cornea without absorption by the corneal tissue. The wavelength of the pulsed laser beam 18 is generally in the range of about 3 μm about 1.9 nm, and preferably between about 400 nm to about 3000 nm. The irradiance of the pulsed laser beam is preferably greater than the threshold for optical breakdown of the tissue. Although a non-UV, ultrashort pulsed laser beam is described in this embodiment, the pulsed laser beam may have other pulse durations and different wavelengths in other embodiments.

The beam may be scanned by selectively moving the focal spot of the beam to produce a structured scan pattern (e.g., a raster pattern, arcs, linear segments, rings, cylinders, a spiral pattern, or the like) of scan spots. The step rate at which the focal spot is moved is referred to herein as the scan rate. Exemplary operating scan rates are between about 10 kHz and about 400 kHz, or at any other desired scan rate. Further details of laser scanners are known in the art, such as described, for example, in U.S. Pat. No. 5,549,632, the entire disclosure of which is incorporated herein by reference.

In one embodiment, scanning mirrors or other optics are employed to angularly deflect and scan one or more input beams. For example, scanning mirrors may be driven by galvanometers where each of the mirrors scans along different orthogonal axes (e.g., an x-axis and a y-axis). A focusing objective having one or more lenses can be used to image the input beam onto a focal plane. The focal spot may thus be scanned in two dimensions (e.g., along the x-axis and the y-axis) within the focal plane. Scanning along the third dimension, i.e., moving the focal plane along an optical axis (e.g., a z-axis), may be achieved by moving the focusing objective, or one or more lenses within the focusing objective, along the optical axis. Thus, a variety of scanned paths or patterns are obtainable from the beam.

A variety of techniques may be used to align the scanned pattern with the eye. In some embodiments, iris registration methodology associated with ablation procedures, such as used for LASIK, marking and/or fiducial techniques used with corneal flap creation, keratoplasty, and the like, and centration can be used to align the incision pattern with the eye. For example, U.S. Pat. Nos. 7,261,415 and 7,044,602, which are herein incorporated in entirety by reference, describe registration techniques to track the position of the eye. Additionally, the alignment reference may vary for different refractive corrections and be based on a variety of ocular features. For example, the alignment reference can be based on the pupil center, the iris boundary, and the like. In one embodiment, the alignment of the scanned pattern accounts for pupil center shift, which may occur as a result of inconsistent iris actuation.

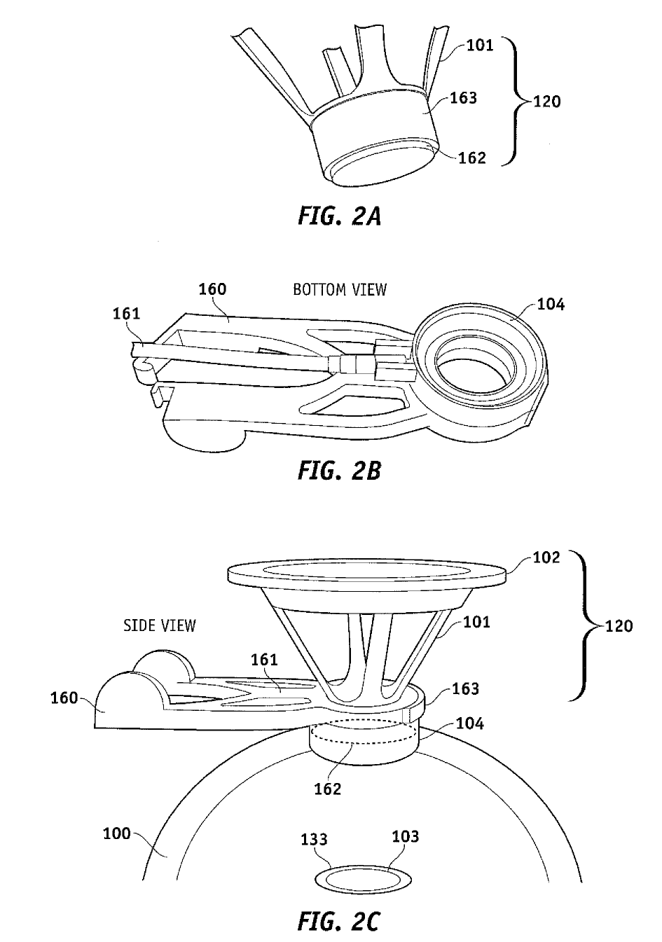

FIGS. 2A-C illustrate several views of the patient interface 120. FIG. 2A shows a distal end of the patient interface cone 101. This embodiment of the patient interface also shows an interface lens 162 which contacts the patient's eye. However, the embodiments of the patient interface without the interface lens are also possible. The interface lens can be rigid, thus conforming the patient's eye to its shape at contact, but it can also be soft, thus inhibiting the deformation of the patient's eye at contact. For example, the interface lens may be replaced by a substantially transparent membrane or film (e.g., derived from transparent plastic, transparent elastomer, transparent vinyl film, transparent polyethylene film, or the like) for contacting the patient's eye. The patient interface cone 101 has a substantially round mating surface 163 for the engagement with the vacuum ring 104, as explained in more detail below.

FIG. 2B illustrates a vacuum ring 104 of the patient interface 120. The distal side of the vacuum ring 104 is shown facing up. A physician can attach the distal side of the vacuum ring to the patient's eye using a gripper unit 160. Tubing 161 fluidically connects the vacuum ring 104 and a syringe (not shown) that can be operated to create a vacuum, thus attaching the vacuum ring 104 to the patient's eye. Thus, the vacuum ring 104 can be a substantially annular eye engagement surface. If the laser light photoalteration is to be performed on the cornea, then the vacuum ring 104 is preferably attached outside of the corneal area of the eye (e.g., contacting the scleral portion of the eye) to inhibit the distortion of the cornea.

FIG. 2C illustrates attaching the patient interface 120 to the patient's eye. A patient interface having the interface lens 162 is shown, but the embodiments of the patient interface without the interface lens are also possible. The patient interface 120 can be positioned on the patient's eye as follows. The vacuum ring 104 is held in contact with the mating surface 163 by the gripper unit 160, thus holding the patient's interface cone 101 and the vacuum ring 104 together. Next, the vacuum ring 104 is brought in contact with the patient's eye, and the vacuum is created by the syringe (not shown) attached to the tubing 161, thus securing the vacuum ring 104 to the patient's eye. Due to the contact of the vacuum ring 104 to the patient's eye and to the patient interface cone 101, the patient's eye is fixated such that the reference surface 102 tracks the movements of the patient's eye. The patient interface 120 can be intended for single-use, having a disposable pre-sterilized suction ring 104 and interface lens 162.

FIGS. 3A-C illustrate an embodiment of the invention with a sequential docking of the ophthalmic measurement system and laser to the patient interface. FIG. 3A shows the patient interface 120 docked to the patient's eye 100. Docking procedure like the one explained in relation to FIGS. 2A-C can be used to attach the patient interface 120 to the eye. Due to a substantially fixed position of the vacuum ring 104 with respect to the patient's eye and the patient interface 120, the reference surface 102 is in a substantially fixed position to the patient's eye, and is now ready to dock with the optical coherence tomographer 110 or the laser 112.

FIG. 3B shows the optical coherence tomographer 110 in a docked position. The patient reference surface 102 and the mating surface on the optical coherence tomographer 110 can be smooth flat surfaces with guiding rails, like, for example, in the IntraLase® Patient Interface by Abbott Medical Optics. Other designs of the precision mating surfaces are possible. When docked to the patient interface 120, an ophthalmic measurement system, for example the optical coherence tomographer 110, maintains a substantially fixed position with respect to the patient's eye. The optical coherence tomographer 110 can acquire the location data on the structures of interest in the patient's eye by, for example, imaging the corneal area or eye lens. Different optical coherence tomographers can be used to acquire the location data. One example is a Visante™ OCT by Carl Zeiss Meditec AG. Another example of an ophthalmic measurement system that can be used to acquire the location data is a WaveScan WaveFront® wavefront aberrometer by Abbott Medical Optics. The location data is made available to the controller 111, which may be an industrial controller or a general purpose computer or other type of controller. The controller 111 generally comprises at least one processor board. Controller 111 may include many of the components of a personal computer, such as a data bus, a memory, input and/or output devices (including a touch screen), and the like. Controller 111 will often include both hardware and software, with the software typically comprising machine readable code or programming instructions for implementing one, some, or all of the methods described herein. The code may be embodied by a tangible media such as a memory, a magnetic recording media, an optical recording media, or the like. Controller 111 may have (or be coupled to) a recording media reader, or the code may be transmitted to the controller by a network connection such as an internet, an intranet, an Ethernet, a wireless network, or the like. Along with programming code, controller 111 may include measurement or other stored data for implementing the methods described herein, and may generate and/or store data that records parameters reflecting the treatment of one or more patients. After acquiring the required location data, the optical coherence tomographer 110 can be undocked from the patient interface 120, thus making the reference surface 102 available to the laser 112 to reference with respect to the patient's eye, as illustrated in FIG. 3C. The controller 111 calculates the laser target data based on the location data generated by the optical coherence tomographer 110. The laser target data represent those portions of the eye tissue which the laser photoalters or otherwise treats during the surgery. FIG. 3C illustrates a flap 116 having a thickness al being created by the laser photoalteration, but other examples are possible, for example, cutting a flap or a hole in the capsular bag 133, photoaltering parts of the eye lens 103 or ablating the cornea, thus changing its thickness σ2. If wanted, the laser target data can be refreshed by undocking the laser 112, docking the optical coherence tomographer 110 or another ophthalmic measurement system, refreshing the location data, and calculating a new set of the laser target data by the controller 111.

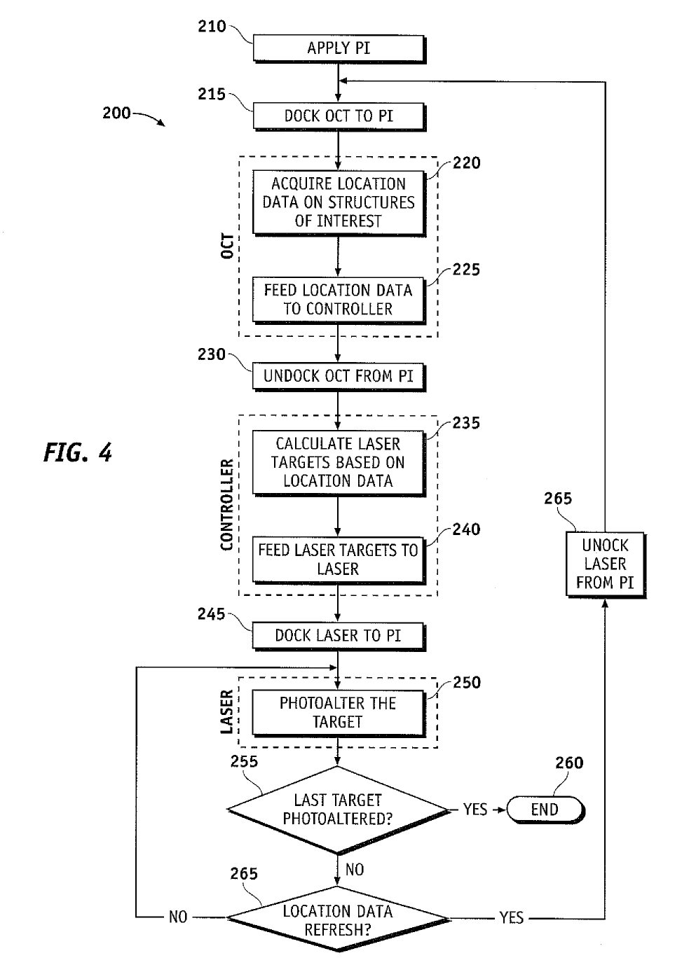

FIG. 4 shows a flowchart of the method 200 that can be used in conjunction with the invention embodiment shown in FIGS. 3A-C. However, it should be understood that many variations of the system shown in FIGS. 3A-C are possible, while still making the method 200 applicable.

At step 210 a patient interface is applied to the patient's eye. An example of applying the patient interface to the patient's eye is illustrated in FIGS. 2A-C, but other examples the patient interface attachment are possible, too. After applying the patient interface to the patient eye, a reference surface is available for referencing the position of the structures of interest in the eye.

At step 215 an ophthalmic measurement system, for instance the optical coherence tomographer, is docked to the patient interface. Since the patient interface maintains its position with respect to the patient's eye, the docked optical coherence tomographer maintains reference to the structures of interest in the eye.

At step 220 an ophthalmic measurement system acquires location data on the structures of interest in the patient's eye. The location data is the position of different tissues in the eye, for example, the cornea and various structures associated therewith (e.g., epithelium, Bowman's layer, stroma, Descemet's membrane, and endothelium), iris, eye lens, or capsular bag.

At step 225 the location data acquired at step 220 are made available to the controller.

At step 230 the optical coherence tomographer is undocked from the patient interface to make the reference surface of the patient interface available to the laser. The reference surface maintains a substantially fixed position to the patient's eye, thus both the ophthalmic measurement system and the laser will also maintain a substantially fixed position to the patient's eye when docked to the patient interface.

At step 235 the controller calculates the laser targets, which are the volumes in the eye to be photoaltered, based on the location data received from the optical coherence tomographer. The laser targets are made available to the laser at step 240.

At step 245 the laser is docked to the patient interface. Thus, the reference surface, which was used to reference the ophthalmic measurement system is now used to reference laser with respect to the structures in the eye.

At step 250 the laser photoalters the laser targets received from the controller. Thus, the tissue of the patient's eye that corresponds to the laser targets is photoaltered by the laser light energy.

At step 255 a check is performed to verify whether all the laser targets have been photoaltered. If more laser targets remain, then they are photoaltered at step 250, followed by repeating the check at step 255. If the last laser target has been photoaltered, then the photoalteration stops at step 260.

The reference surface of the patient interface may not precisely follow the location of the structure of interest in the patient's eye because of the deformation of the eye. Therefore, a refresh of the location data may be desired, as shown at step 255. To refresh the location data, the laser is undocked from the patient interface at step 265, followed by docking the optical coherence tomographer at step 215. The optical coherence tomographer is now ready to acquire additional location data, thus refreshing the location data on the structures of interest in the patient's eye.

The method as described above with reference to FIG. 4 can have many variations. For example, calculation of the laser targets can be performed while the optical coherence tomographer is still docked to the patient interface, or after the laser is docked to the patient interface. The laser targets can be fed to the laser before or after its docking with the patient interface. Furthermore, after one or more laser targets are calculated and photoaltered, the controller may calculate another one or more laser targets, and feed them to the laser for the photoalteration. Rather than ablating a target, the target may be simply heated so as to induce a change in shape of the tissue, to induce necrosis, or the like. Other types of the ophthalmic measurement system can be used instead of the optical coherence tomographer. Many other variations are possible without deviating from the spirit of the disclosed invention.

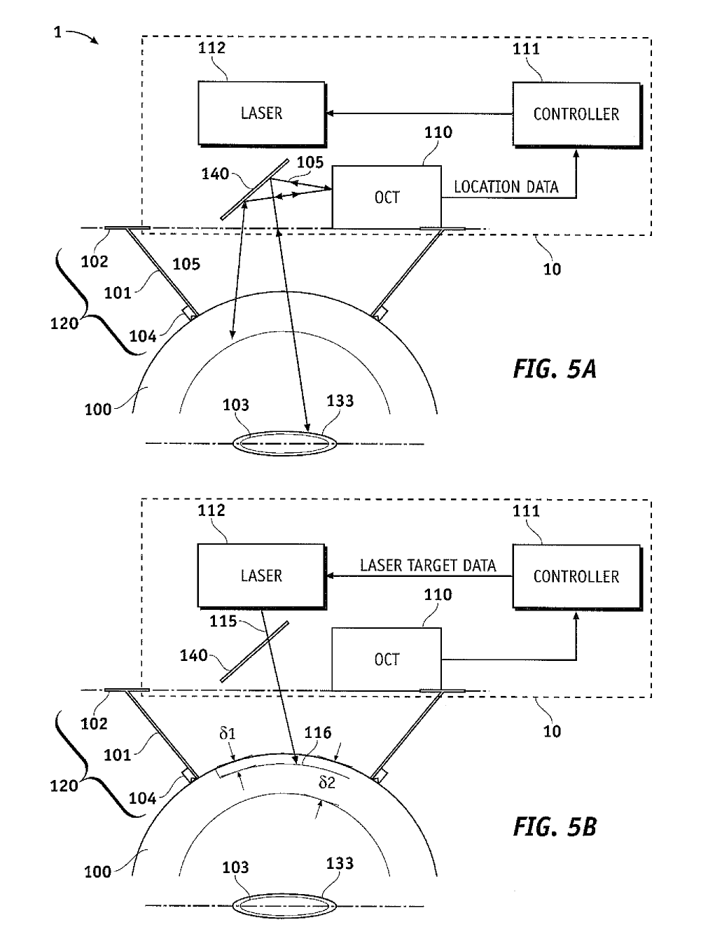

FIGS. 5A-B illustrate an embodiment of the invention having a simultaneous docking of the optical coherence tomographer and the laser to the patient interface. In this embodiment, the optical coherence tomographer and laser are coupled together in an integrated system, for example. FIG. 5A illustrates that the optical coherence tomographer 110, controller 111, and laser 112 can be connected in a photoalteration apparatus 10. If the locations of the optical coherence tomographer and the laser are known relative to the photoalteration apparatus, then docking the photoalteration apparatus or, for example, docking only the optical coherence tomographer to the patient interface would suffice to reference both the optical coherence tomographer and the laser to the patient eye. A person having ordinary skill in the art would know of many ways of designing a photoalteration apparatus to connect the optical coherence tomographer, controller, and laser such that the relative position of the optical coherence tomographer and the laser is known. The photoalteration apparatus 10 can have a beamsplitting mirror 140 that reflects the light having a wavelength below a certain threshold, while transmitting the light above that wavelength threshold. Thus, the visible light 105 emitted by the optical coherence tomographer 110 is reflected off the beamsplitting mirror 140 into the interior of the patient's eye 100, and back to the optical coherence tomographer 110, which creates the location data, and makes them available to the controller 111.

FIG. 5B illustrates the photoalteration of the targets in the patient's eye. Based on the location data provided by the optical coherence tomographer 110, the laser target data are calculated by the controller 111, and are made available to the laser 112. Since the laser 112 references with the optical coherence tomographer 110 within the photoalteration apparatus 10, there is no need to dock the laser directly to the patient interface in order to reference the laser target data to the structures of interest in the patient's eye. The beamsplitting mirror 140 transmits the laser light 115 because of its long wavelength, and the laser light 115 photoalters the tissue in the patient's eye. FIG. 5B illustrates a corneal flap made by the laser photoalteration, but other targets are also possible, including, for example, ablating the portions of the cornea, eye lens, or capsular bag.

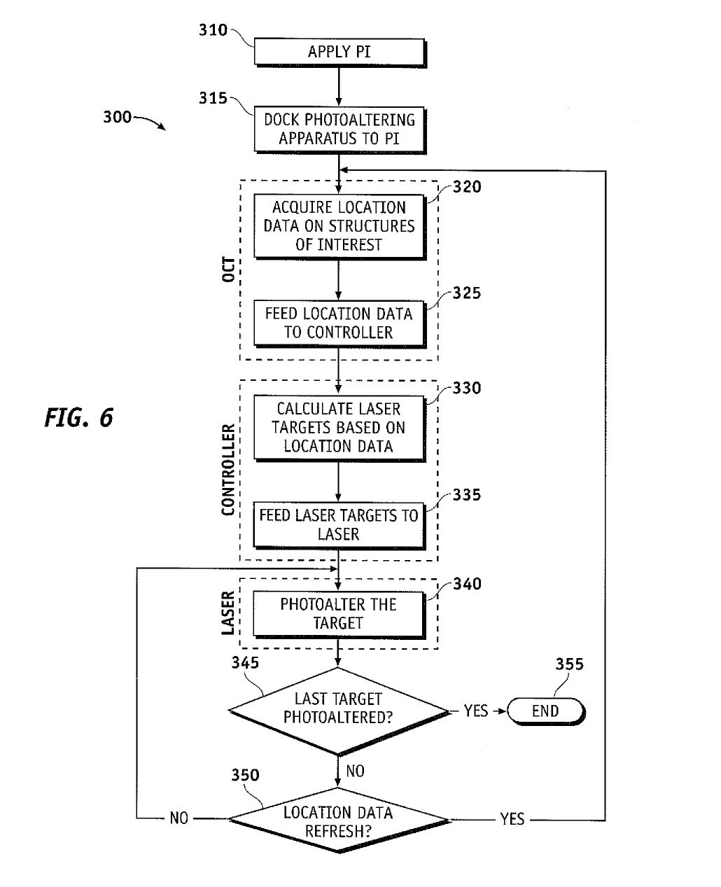

FIG. 6 shows a flowchart of a method 300 that can be used in conjunction with the invention embodiment shown in FIGS. 5A-B.

At step 310 a patient interface is applied to the patient's eye. The application of the patient interface to the patient eye makes the reference surface 102 available for referencing the position of the structures of interest in the eye.

At step 315 the photoalteration apparatus is docked to the patient interface. As explained in conjunction with FIG. 5A, it suffices to dock just one component, for instance the optical coherence tomographer, to the patient interface, since the relative position of the laser to the optical coherence tomographer is known. Alternatively, the frame containing the elements of the photoalteration apparatus can be docked to the patient interface thus maintaining the reference to the structures of interest in the eye.

At step 320 the optical coherence tomographer acquires location data on the structures of interest in the patient's eye. The beamsplitting mirror can be used to reflect the light to the patient's eye and back to the optical coherence tomographer. The location data can be the position of different tissues in the eye, for example, the cornea, eye lens, or capsular bag.

At step 325 the location data acquired at step 320 are made available to the controller, which calculates the laser targets, i.e. the volumes to be photoaltered, at step 330. The laser targets are made available to the laser at step 335. As explained above, docking of the photoalteration apparatus references both the optical coherence tomographer and the laser to the reference surface 102.

At step 340 the laser ablates the laser targets received from the controller. Thus, the tissue of the patient's eye that corresponds to the laser targets is ablated by the laser light energy.

At step 345 a check is performed to verify if all the laser targets have been photoaltered. If some laser targets remain, then they are photoaltered at step 340, followed by repeating the check for the remaining laser targets at step 345. If the last laser target has been photoaltered, then the photoalteration stops at step 355.

A refresh of the location data, which may be desired to account for the deformation of the eye, is shown in step 255. With this embodiment of the invention the refresh of the location data does not necessitate additional docking and undocking of the laser or the optical coherence tomographer.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments described in the specification. As one of ordinary skill in the art will readily appreciate from the disclosure of the present invention, many variations of the disclosed systems and methods are possible without deviating from the spirit of the invention.

What is claimed is:

1. A laser surgery system for treatment of an eye, the system comprising:
a patient interface comprising a reference surface and an eye-engagement surface configured to couple to and fixate the eye;
an ophthalmic measurement system that, in use, generates location data corresponding to internal surfaces of the eye, the measurement system configured to couple with the reference surface;
a laser configured to couple with the reference surface; and
a controller coupled with the ophthalmic measurement system and the laser, the controller configured to process location data from the ophthalmic measurement system and to compute laser target data so that the laser photoalters an internal target within the eye in response to the location data;
wherein the patient interface further comprises a flexible membrane coupled to the eye-engagement surface, the flexible membrane configured to permit a beam transmission from the laser therethrough and further configured to contact an anterior surface of the eye and conform the flexible membrane to the anterior surface of the eye.

2. The system of claim 1, wherein the eye engagement surface comprises a vacuum ring configured to be positioned peripheral to a treated area of the eye by the laser, thus inhibiting distortion of the internal target within the eye.

3. The system of claim 1, wherein the ophthalmic measurement system is selected from a group consisting of an optical coherence tomographer, a wavefront aberrometer, a topographer, and a combination thereof.

4. The system of claim 1, wherein the ophthalmic measurement system generates location data corresponding to internal surfaces from a group consisting of a corneal flap, a cornea, a capsular bag, a lens, and a combination thereof.

5. The system of claim 1, wherein the controller is further configured to process location data from the ophthalmic measurement system and to compute laser target data so that the laser photoalters an internal corneal volume of the eye in response to the location data.

6. The system of claim 1, wherein the controller is further configured to process location data from the ophthalmic measurement system and to compute laser target data so that the laser photoalters a corneal flap of the eye.

7. The system of claim 1, wherein the flexible membrane is selected from the group consisting of a transparent plastic, a transparent elastomer, a transparent vinyl film, and a transparent polyethylene film.

8. A system for laser surgery treatment of an eye that, in use, generates a location data corresponding to internal surfaces of the eye using an ophthalmic measurement system, calculates internal targets within the eye based on the location data using a controller, and photoalters the internal targets within the eye using a laser, the system comprising:
a patient interface having:
a reference surface configured to couple with the laser and the ophthalmic measurement system, and
an eye-engagement surface configured to attach with the eye;
wherein the patient interface further comprises a flexible membrane coupled to the eye-engagement surface, the flexible membrane configured to permit a beam transmission from the laser therethrough and further configured to contact an anterior surface of the eye and conform the flexible membrane to the anterior surface of the eye.

9. The system of claim 8, wherein the eye-engagement surface is a substantially annular area outside of a treated area of the eye, thus inhibiting distortion of the internal target within the eye.

10. The system of claim 8, wherein the flexible membrane is selected from the group consisting of a transparent plastic, a transparent elastomer, a transparent vinyl film, and a transparent polyethylene film
